(19) United States
(12) Patent Application Publication
(10) Pub. No.: US 2021/0274034 A1
(43) Pub. Date: Sep. 2, 2021

(54) SYSTEMS AND METHODS FOR DELIVERING TIMELY PERSONALIZED INFORMATION OF VULNERABLE PERSONS TO AUTHORITIES AND/OR CAREGIVERS

(71) Applicant: VITALS AWARE SERVICES, Eden Prairie, MN (US)

(72) Inventors: Steve Mase, Minneapolis, MN (US); Janee Harteau, Lakewood Ranch, FL (US); Justin Grammens, St. Paul, MN (US); Robert Zink, Baldwin, WI (US)

(73) Assignee: VITALS AWARE SERVICES, Eden Prairie, MN (US)

(21) Appl. No.: 17/189,000

(22) Filed: Mar. 1, 2021

Related U.S. Application Data
(60) Provisional application No. 62/984,105, filed on Mar. 2, 2020.

(51) Int. Cl. H04M 1/72421; H04W 4/80; H04W 4/02; H04W 4/021; H04M 1/72457

(52) U.S. Cl. CPC H04M 1/72421; H04W 4/80; H04M 1/72457; H04W 4/021; H04W 4/023

(57) ABSTRACT

Systems and methods for delivering timely personalized information about vulnerable persons to authorities and/or to caregivers. This may provide critical situational information in real or near real time to authorities and/or to caregivers in order to help increase public safety, reduce risks and/or improve the quality of life for vulnerable persons and parties responsible for the safety and well-being of the vulnerable persons.

### CROSS REFERENCE TO RELATED APPLICATIONS

This application claims the benefit of copending U.S. Provisional Application Ser. No. 62/984,105, filed Mar. 2, 2020, and entitled “SYSTEMS AND METHODS FOR DELIVERING TIMELY PERSONALIZED INFORMATION OF VULNERABLE PERSONS TO AUTHORITIES AND/OR CAREGIVERS”, which is incorporated herein by reference.

### TECHNICAL FIELD

The present disclosure relates to systems and methods for delivering timely personalized information of vulnerable persons to authorities and/or caregivers.

### BACKGROUND

A significant percentage of the world's population lives with some sort of disability that poses very real challenges to daily life. Often these disabilities are misunderstood or are invisible, or nearly so, to the public and to strangers. Many of these disabilities are mental illnesses, such as schizophrenia, bipolar disorder, anxiety, depression, Alzheimer's, or dementia. Others are purely physical, such as epilepsy, diabetes, deafness, blindness, muteness, or medication effects. And some are both mental and physical, such as autism, fetal alcohol disorder, post-traumatic stress, or attention deficit/hyperactivity. It can be difficult for people experiencing these disorders to live a normal and safe life, in part because some of the time they look and act “normal” to others, who do not know of or suspect the disabilities.

When such vulnerable people encounter authority figures, chances are heightened that the encounters will take a negative turn. This is particularly true with first responders, such as law enforcement, fire fighters and emergency medical personnel. They are trained to make quick judgments in high-stress situations. Much of their training is one-size-fits-all and is based on rapid assessment of circumstances followed by rapid reaction. These circumstances typically do not allow time for reflection or nuance. The situational stress, or escalated state, may make the vulnerable parties react in ways that can appear threatening or uncooperative to responders. That escalates the stressful encounters.

A Pew Research Center study of a recent multi-year period across the United States analyzed all law enforcement-citizen encounters that resulted in injury or death. The study indicated that approximately half of those encounters involved people with invisible disabilities. To illustrate, responders may think that a deaf person is ignoring police commands, that a diabetes sufferer is inebriated, or that a person with schizophrenia is high on drugs. An autistic teen-ager's stress-induced attempt to run can be mistaken as a flight from justice. These misunderstandings can lead to endings that can be tragic for all parties.

Responders have taken steps to reduce these risks. Many law enforcement agencies have put their officers through specialized training to recognize vulnerabilities, and to deal with vulnerable persons in specialized ways. Many agencies also have emphasized community policing efforts, so that geographically assigned patrol officers know their neighborhoods and have a better chance of recognizing vulnerable persons.

Daily life also poses risks for children and for persons with disabilities that are visible, such as advanced age, illness, or physical impairment. Without constant supervision, they may wander away, become lost, or stray into dangerous situations.

As a result, law enforcement leadership is frustrated, caregivers of such vulnerable persons are fearful, and vulnerable people remain at risk. What is needed are systems and methods for delivering timely personalized information about vulnerable persons to authorities and/or caregivers to help increase public safety, reduce risks and/or improve the quality of life for vulnerable persons and parties responsible for the safety and well-being of the vulnerable persons.

### SUMMARY

The present disclosure relates to systems and methods for delivering timely personalized information about vulnerable persons to authorities and/or to caregivers. In some cases, this may provide critical situational information in real or near real time to authorities and/or to caregivers in order to help increase public safety, reduce risks and/or improve the quality of life for vulnerable persons and parties responsible for the safety and well-being of the vulnerable persons.

In some instances, the system may include a network based on a mobile communications platform utilizing tightly integrated multiple specialized software applications and hardware devices developed to work together to detect and identify vulnerable persons, to match the vulnerable persons with unique high-value personalized information, and to deliver the high-value information to authorized recipients in real or near-real time. For first responders, emergency personnel and caregivers in educational and institutional environments, the disclosure identifies the vulnerable parties to the responder before the engagement, and provides critical information prior to the engagement. The better-informed responder or institutional party is thus able to adapt situational management to increase the chances of a positive outcome.

In some cases, the vulnerable persons may carry a device such a mobile phone with location monitoring capabilities. The location monitoring capabilities may include GPS, cellular triangulation, or Bluetooth or Wifi connectivity to a gateway with a known location, etc. The system may generate an electronic notification in real or near real time when the vulnerable person has wandered away from one or more pre-defined areas and/or has entered into one or more pre-defined restricted areas. In some cases, this may trigger a notification to a caregiver's device and/or to the authorities. When a caregiver's device is notified that the vulnerable person has wandered away, the caregivers may be given the option to send a signal through the present system to activate the vulnerable person's device, causing instantaneous transmission notifying authorized recipients of the vulnerable person's location.

In some cases, a server or similar device may be used as a central platform that controls the overall functionality of the system. When so provided, the server may be operatively coupled to one or more end user devices. Communications can occur across one or more communication channels such as Ethernet, the Internet, Cellular, Wifi, Bluetooth, and/or any combination thereof. In some cases, the end user devices may be mobile devices such as mobile phones, tablet computers, laptop computers and/or portable beacons, although desktop computers and/or other similar devices may be used where appropriate. In some cases, most components of the system (except perhaps the location monitoring capabilities) may be always on, sometimes operating in background mode until an alert or an information delivery is warranted.

An example method may include using a server or the like to deliver timely personalized information of vulnerable persons to authorities and/or caregivers. This example method may include receiving at the server an identifier for a vulnerable person from a first remote device that has come within range of and detected a beacon of the vulnerable person. The first remote device may be a mobile device carried by a first responder, and the beacon may be a mobile device carried or worn by the vulnerable person. In some cases, the beacon repeatedly transmits a beacon identifier signal using Bluetooth Low Energy (BLE), and the first remote device receives the beacon identifier signal when the first remote device is within range of the beacon. The identifier that is received at the server may be transmitted by the first remote device and may be based at least in part on the beacon identifier signal. The server may use the identifier to identify the vulnerable person, and identify a profile for the identified vulnerable person by accessing a vulnerable person database. The profile may have been previously provided by a second remote device. In some cases, the second remote device may be a device of a caregiver of the vulnerable person, such as a mobile device, a laptop, a desktop, a tablet or any other device of the caregiver. The server may provide at least part of the identified profile to the first remote device (e.g. of the first responder) that has come within range of and detected the beacon of the vulnerable person. In some cases, a notification may be sent to the second remote device (e.g. of the caregiver) when the first remote device has come within range of and detected the beacon of the vulnerable person.

Another example method includes receiving an identifier for a vulnerable person from a first remote device that has come within range of and detected a beacon of the vulnerable person. The first remote device may be a mobile device carried by a first responder, and the beacon may be a mobile device carried or worn by the vulnerable person. The illustrative method may use the identifier to identify the vulnerable person, and identify a profile for the identified vulnerable person. At least part of the profile may be received from a second remote device, such as a device of a caregiver of the vulnerable person. At least part of the identified profile may be provided to the first remote device that has come within range of and detected the beacon of the vulnerable person.

In addition, and regardless of whether the first remote device has come within range of and detected the beacon of the vulnerable person, the illustrative method may identify when the beacon has crossed a predefined geofence, and may send a notification to the second remote device when the beacon has crossed the predefined geofence. This may alert the caregiver that the vulnerable person has wonder off. In some cases, the notification to the second remote device includes location information of the beacon, and thus the location of the vulnerable person. In some cases, the caregiver can transmit the location to a 9-1-1 dispatch center notifying the authorities of the location of the vulnerable person.

Another example method includes receiving an identifier for a vulnerable person from each of a plurality of responder remote devices over time that have encountered the vulnerable person by coming within range of and detecting a registered beacon of the vulnerable person. The illustrative method includes using the identifier to identify the vulnerable person on each encounter, and identify a profile for the identified vulnerable person on each encounter. In some cases, at least part of the profile has been provided by a caregiver remote device. The method may further include providing at least part of the identified profile to the corresponding responder remote device on each encounter. The method may also include sending a description and/or history of the encounters to the caregiver remote device. In some cases, the description and/or history of encounters is sent to the caregiver remote device upon receiving a request from the caregiver remote device. Also, the history of encounters can be filtered to only send part of the encounters to the caregiver remote device.

### BRIEF DESCRIPTION OF THE DRAWINGS

FIG. 1 is a schematic block diagram of networked components of an illustrative system suitable for delivering timely personalized information of vulnerable persons to authorities and/or caregivers;

FIG. 2 is a schematic block diagram of the system of FIG. 1 showing illustrative Caregiver Node interactions within networked components of the system;

FIG. 3 shows illustrative screen shots of an application program (app) running on a first responder's mobile device;

FIG. 4 is a schematic block diagram of the system of FIG. 1 showing illustrative tethering interactions within networked components of the system;

FIG. 5 shows illustrative screen shots of an application program (app) running on a caregiver's mobile device showing an illustrative tethering notification;

FIG. 6 is a schematic block diagram of the system of FIG. 1 displaying interactions within networked components of the system during activation of the “historical display” function;

FIG. 7 show screen shots of an illustrative application program (app) running on a caregiver's mobile device showing an illustrative “historical display;”

FIG. 8 is a schematic block diagram of the system of FIG. 1 showing illustrative interactions among networked components during activation of geofencing;

FIG. 9 show screen shots of an illustrative application program (app) running on a caregiver's mobile device showing an illustrative geo-fencing interaction feature;

FIG. 10 is a schematic block diagram of the system of FIG. 1 showing interaction among networked components of the system during implementation of the “god view” functionality;

FIG. 11 is a schematic block diagram of the system of FIG. 1 showing illustrative responder interactions via the Responder Node;

FIG. 12 is a schematic block diagram of the system of FIG. 1 showing illustrative Beacon Node interactions among networked components of the system;

FIG. 13 is a schematic block diagram of the system of FIG. 1 showing illustrative Smart Node interactions among networked components of the system; and

FIG. 14 is a schematic block diagram of the system of FIG. 1 showing illustrative Gateway Node interactions among networked components of the system.

### DESCRIPTION

The terminology used herein is for the purpose of describing particular embodiments only and is not intended to be limiting of the disclosure. As used herein, the term “and/or” includes any and all combinations of one or more of the associated listed items. As used herein, the singular forms “a,” “an,” and “the” are intended to include the plural forms as well as the singular forms, unless the context clearly indicates otherwise. Also herein, the use of “comprises,” “comprising,” “composes,” and/or “composing,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this disclosure belongs. All terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the disclosure, it should be understood that a number of techniques and steps are disclosed. Each of these has person benefit and each can also be used in conjunction with one or more, or in some cases all, of the other disclosed techniques. Accordingly, for the sake of clarity, this description will refrain from repeating every possible combination of the person steps in an unnecessary fashion. Nevertheless, the specifications and claims should be read with the understanding that such combinations are entirely within the scope of the disclosure and the claims.

The present disclosure is to be considered as exemplifying the disclosure, and it is not intended to limit the disclosure to the specific embodiments illustrated by the figures or descriptions herein.

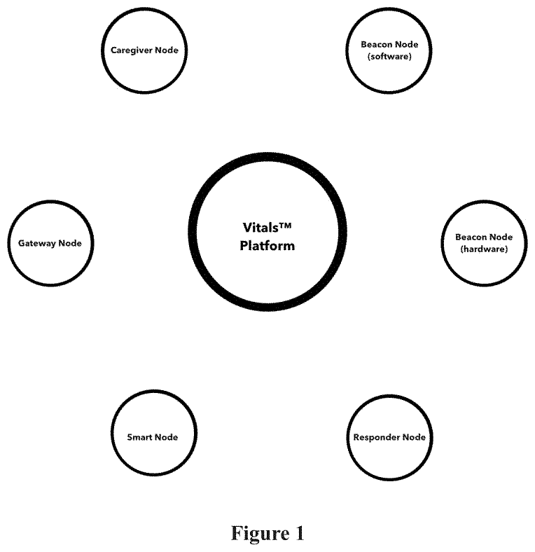

FIG. 1 is a schematic block diagram of networked components of an illustrative system suitable for delivering timely personalized information of vulnerable persons to authorities and/or caregivers. The illustrative system may include a tightly integrated system of software applications and hardware devices. In some cases, the system's central and controlling role rests with the Vitals™ Platform, and with the exception of certain location-based communications, all Network communications flow through the Vitals™ Platform. The Vitals™ Network may utilize peer-to-peer communications with storage in the Cloud.

In some cases, the system architecture may be considered a “Network” that has a number of interconnected “Nodes,” which may be referred to as the Vitals™ Network. In some cases, the Vitals™ Network may include a central Vitals™ Platform that functions as a central node. The Vitals™ Network may also include a number of remote nodes that are operatively coupled to the central node. Each node may include both a hardware device and associated software that implements the function of the particular node.999

In some cases, each caregiver within the Vitals™ Network may be given a Caregiver Node. The Caregiver Node may be, for example, a mobile phone running a Vitals™ Caregiver software application. The Caregiver may use the Caregiver Node to setup a secure account with the Vitals™ Platform and enter and upload to the Vitals™ Platform critical information about the vulnerable person in the caregiver's charge (the “Profile”).

The vulnerable person may be given a Beacon Node, which may be a software application resident on the vulnerable person's smart phone and/or may be a small physical transmitting device. Each Beacon Node may be “paired” with its applicable caregiver account already created in the Vitals™ Platform. Each Beacon Node transmits signals uniquely identifying the person carrying that Beacon Node. In some cases, the signals are coded Bluetooth Low Energy (BLE) signals that can be received as far away as 80 feet. In some cases, the signals can only be decoded by parties that are registered with the Vitals™ Network and authorized by the Vitals™ Platform to decode the signals, such as the vulnerable person's caregiver and/or first responders such as police, fire, EMS, etc. The may help alleviate data privacy concerns related to the vulnerable person.

First responders and other authorized parties may carry a Vitals™ Responder Node registered with the Vitals™ Network. In some cases, the Vitals™ Responder Node may be, for example, a mobile phone running a Vitals™ Responder software application. In some instances, the Vitals™ Responder software application is designed to remain always on in background. When a responder comes within approximately 80 feet of a Beacon Node (e.g. BLE is used), the Vitals™ Platform notifies the responder via the Vitals™ Responder software application and provides the available critical information to responder in a real or near real-time manner. This is designed to occur before the actual encounter occurs between responder and vulnerable person, allowing the responder time to view the critical information in order to better deal with the encounter.

The Vitals™ Platform documents each encounter between a Responder Node and a Beacon Node of each vulnerable person, and in some cases, and notifies the relevant Caregiver Node.

The Vitals™ System may also provide other functionalities intended to increase safety by providing critical information in real time or near real time. These other functionalities may include, but are not limited to, (a) a “tethering” functionality, which may include providing real-time notifications to the caregiver when a pre-determined distance between a Caregiver Node and a relevant Beacon Node is exceeded, (b) a “geofencing” functionality, which may include allowing the caregiver to select or map one or more predetermined geographic areas and to set up corresponding location rules whereby the Vitals™ System then delivers notifications in real time or near real time to the corresponding Caregiver Node when the relevant Beacon Node enters a restricted geographic area or leaves a safe geographic area, (c) a “Smart Node” functionality, which may include adding “Smart Nodes” to the Vitals™ System that are designed for use in fixed locations to compile information obtained from nearby Beacon Nodes and to retransmit the compiled information to responders, much as a Beacon Node would, and (d) a “Gateway Node” functionality, which may include adding one or more “Gateway Nodes” to the Vitals™ System, with each Gateway Node designed for a fixed location use to gather information about nearby activities, with rules-based handling and communication of the information. Tethering and geofencing may be controlled by the relevant Caregiver Node, which in some cases can customize the geofencing criteria. Smart Nodes and Gateway Nodes may be always-on physical devices operatively coupled to the Vitals™ Platform. Smart Nodes may be designed for use in fixed population settings such as group homes. Gateway Nodes may be designed for use in public areas with transitory populations, such as at or near stadiums, shopping malls, hospitals, and/or any other suitable location.

In addition to its coordinating function, the Vitals™ Platform may be used to assemble and process information derived from all remote nodes of the Vitals™ Network. This information may assist various parties in their efforts to increase public and personal safety for vulnerable persons. In some case, the Vitals™ Platform may be implemented in the Cloud. In some cases, the Vitals™ Platform may be hosted on one or more servers in the Cloud, and to facilitate scaling the Vitals™ System, the Vitals™ Platform may be implemented in one or more virtual machines in the Cloud. These are just some examples. Data storage and security may be enhanced by encrypting all stored data and communication in the Vitals™ Network.

In the illustrative system shown in FIG. 1, the Vitals™ Platform plays a central and controlling role in operation of the Vitals™ System. In some cases, the Vitals™ Platform manages electronic interactions with the remote Nodes (e.g. the Responder Node, the Caregiver Node, the Beacon Node, the Smart Node and/or the Gateway Node, when present) to create and manage customer accounts, enable pairing of appropriate Node components, receive and transmit instructions to and from the remote Nodes, store in the Cloud all information derived from Network activities, and use that information to generate analyses and reports required by users and customers. Some illustrative electronic interactions are described in more detail below.

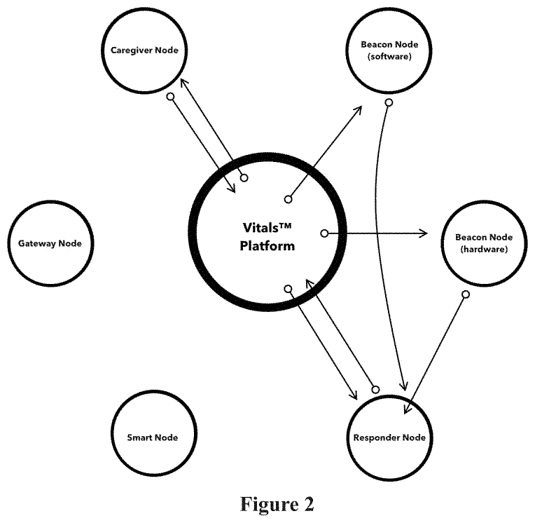

FIG. 2 is a schematic block diagram of the system of FIG. 1 showing illustrative Caregiver Node interactions within the networked components of the system via the Vitals™ Caregiver Node. As the name implies, the Vitals™ Caregiver Node is designed for use by a caregiver, such as a parent or guardian, who is responsible for a vulnerable person. The Caregiver Node may include, for example, a mobile phone running a Vitals™ Caregiver software application. The Vitals™ Caregiver software application may be created in multiple versions for all major operating systems of smart phones, and may be made available at suitable app stores on the Internet. The Caregiver Node may provide bidirectional communication with other nodes in the Vitals™ Network, all of which may be encrypted or otherwise encoded for security. The Vitals™ Caregiver software application may be always on (so long as the host mobile device is on), and may always to be searching for other components of the Vitals™ Network.

In some cases, The Caregiver Node may allow the caregiver to create and manage a Profile that is unique to the vulnerable party. Although the Caregiver Node may be implemented using a mobile device, it is contemplated that a desktop computer may be used for creating or editing the Profile and/or performing other functions including administrative functions. Utilizing a forms-driven interface provided to the user by the Vitals™ Platform in order to create a structured database, the caregiver is able to enter personalized data about the vulnerable party that, in aggregate, comprise the Profile. This Profile may include, but is not limited to, the vulnerable person's name, nickname (if any), photo, physical description, medical condition(s), medications, behavioral triggers, de-escalation techniques, contact information for one or more caregivers, and caregiver-generated custom messages intended for use by the Vitals™ Responder Node. The caregiver also can use the Caregiver Node to upload an audio and/or video file that can be accessed via, for example, the Vitals™ Responder Node. The uploaded audio and/or video files may be played by the Responder Node prior to or during an encounter with the vulnerable person, providing a tool for calming the vulnerable person or defusing a tense situation. Personalized information in the Profile may be stored in the Vitals™ Platform and made available to any of a plurality of Responder Nodes registered with the Vitals™ Network. It is contemplated that the Profile can be operational with partial information.

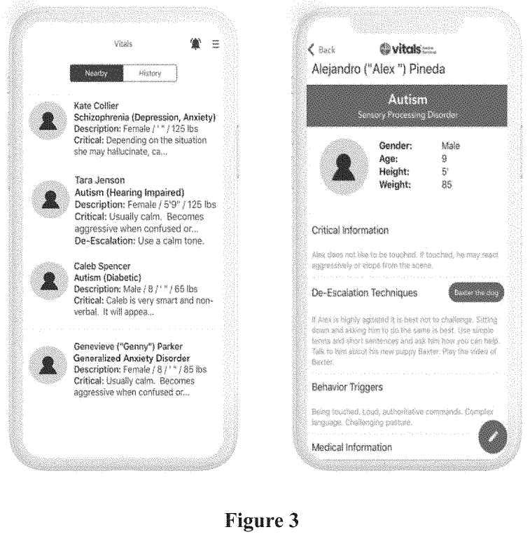

FIG. 3 shows screen shots of an illustrative application program (app) running on a responder's mobile device (e.g. police, fire, EMT, etc.) showing the personalized information of a vulnerable person. This information may be automatically displayed on the responder's mobile device when, for example, the responder's mobile device comes within range (e.g. BLE range) of a beacon node of the vulnerable person.

The Caregiver Node may be used to create each Profile in a secure digital environment maintained by the Vitals™ Platform. Caregiver access may be protected by a unique and secure password assigned as part of account setup and maintained by the caregiver. The caregiver is able to change the Profile at any time, including the password, information additions or deletions. In a more quotidian circumstance, the caregiver might temporarily alter the Profile due to transient events. For example, if the vulnerable person misses medications one day, or runs away from the caregiver after an autistic meltdown, the caregiver may change the Profile temporarily to alert any participating responders to the immediate circumstances. To maintain privacy, all Profile information may be accessible to and created solely by the caregiver, and no other party may be authorized to change or delete the Profile. In some cases, there is no limit to the number or frequency of changes to the Profile that can be made by the caregiver.

The Caregiver Node may be configured to communicate with the Vitals™ Platform via, for example, WiFi, 4/5G and/or LTE cellular channels. In some cases, the Caregiver Node may monitor availability of WiFi and cellular signals and automatically selects the strongest signal (e.g. highest signal-to-noise ratio). In some cases, the Caregiver Node may request that the Vitals™ Platform display on the caregiver's mobile device a live, real-time or near real-time digital map showing recent and currently active Responder Node encounters with Beacon Nodes carried by the caregiver's vulnerable charge. “Encounters,” as defined and recorded in the Vitals™ Platform may include times and locations when Vitals™ Responder Nodes detect the proximity of the caregiver's charge (e.g. within BLE range of 80 feet or less).

When a user account is created by the caregiver, the creator's Caregiver Node may be assigned and “paired” with one or more Beacon Nodes. This pairing, accomplished by the Vitals™ Platform following instructions from the Caregiver Node, enables certain functionalities that are linked to and controlled by the Caregiver Node. The pairing may occur with the assigning of a unique code identifying the Beacon Node itself as well as the vulnerable person to whom the Beacon Node was provided. The pairing may occur from time to time when a new or replacement beacon is delivered. In some cases, the Caregiver Node can be paired with multiple Beacon Nodes. Pairing with a new or replacement Beacon Node can be accomplished by the caregiver through the Caregiver Node, and/or executed centrally from the Vitals™ Platform.

Separate from receiving and decrypting the signals, whether received via WiFi or cellular channels, the Caregiver Node may also repeatedly transmit a unique identifying signal (e.g. at a frequent regular interval) using such as (but not limited to) a Bluetooth Low Energy (“BLE”) signal. This may occur in background mode of the Caregiver Node.

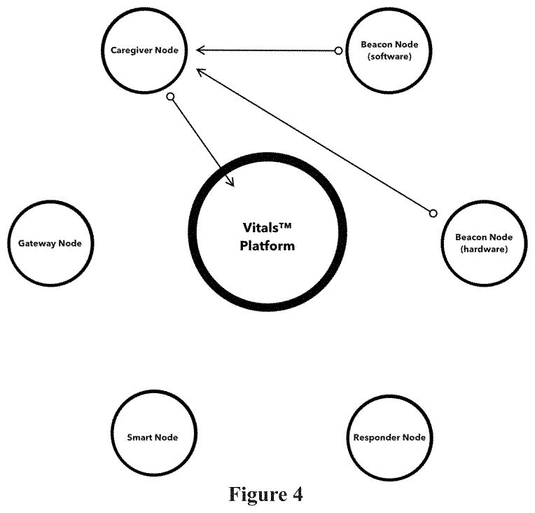

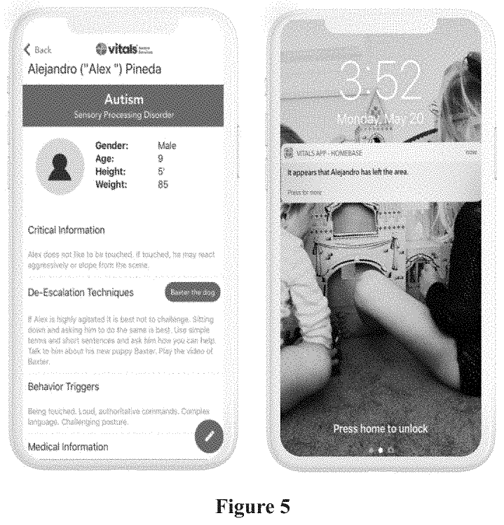

Once paired with a Beacon Node, the Caregiver Node may detect the nearby presence of a Beacon Node that is paired with the relevant Caregiver Node. The Caregiver Node can be used to switch on (or off) a process that sends an alert to the caregiver's smart phone if a distance between the two devices becomes so great that signals (BLE signals) between them cease to be detected. This functionality may be referred to as “tethering.” The resulting alert can be manifested as an audible tone, a vibration, a screen display, or a combination of those methods, the choice of which may be under the Caregiver Node's control. The alert may also be delivered to the Central Vitals™ Platform. FIG. 4 is a schematic block diagram of the system of FIG. 1 showing illustrative tethering interactions of various components with the networked system. FIG. 5 shows screen shots of an illustrative application program (app) running on a caregiver's mobile device showing an illustrative tethering notification.

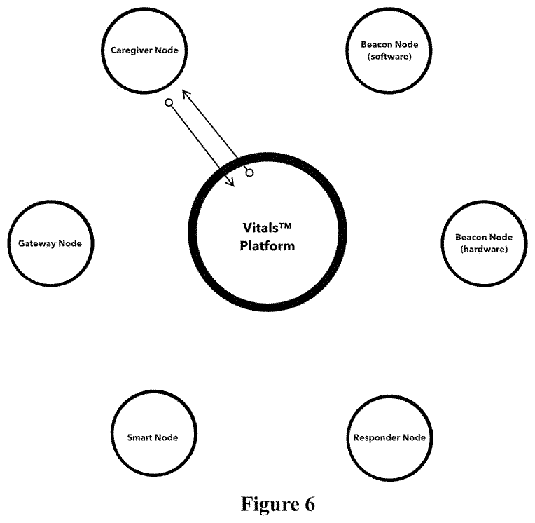

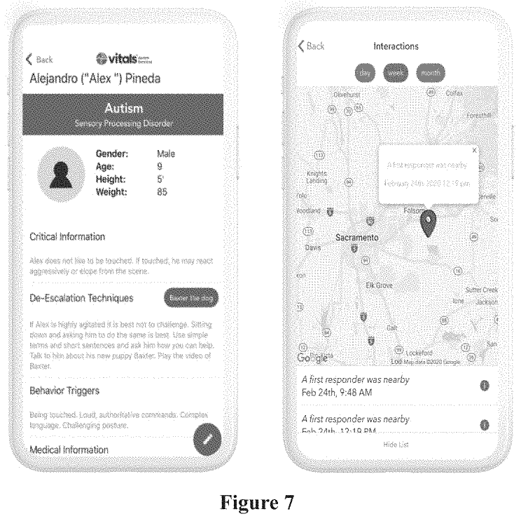

The Caregiver Node may provide a function that can be powered on by the caregiver to replicate certain of the functions of a Responder Node. For example, when powered on, the caregiver may be provided with a visual display of the various encounters between the relevant Beacon Node of the vulnerable person in the caregiver's charge and all other remote Nodes of all types in the Vitals™ Network. In some cases, the display of such information may be under the control of the Caregiver Node and in some cases may be controlled to show current encounters, total encounters during the previous 24 hours, total encounters during the previous week, total encounters during the previous 30 days, and/or total encounters during the previous year. These are just examples. Such functionality may be referred to as “historical display” functionality. In some cases, the historical display visual presentation may use different color coding to differentiate among Responder Nodes, Caregiver Nodes, and Beacon Nodes, but this is not required. FIG. 6 is a schematic block diagram of the system of FIG. 1 indicating “historical display” interactions among components of the networked system. FIG. 7 shows screen shots of an illustrative application program (app) running on a caregiver's mobile device showing an illustrative historical display of interactions. In some cases, the Vitals™ Platform may have the ability to suppress from historical display interactions the current, real-time locations of Responder Nodes. This may provide enhanced security to responder personnel.

The Caregiver Node may also provide notification functionalities utilizing geofencing capabilities. The geofencing functionalities selected by the Caregiver Node may become part of a relevant Profile. The caregiver may enter via the Caregiver Node a location via street address, latitude/longitude coordinates, place name and/or any other suitable location. This information may be transmitted to the Vitals™ Platform, which may respond to the Caregiver Node with a list from which the user can select and verify the chosen location. The verification may be transmitted to the Vitals™ Platform, which may respond with a map displaying the chosen location. In some cases, the Caregiver Node can be used to manipulate the map, adjusting image size and location. When the caregiver has selected a location, the Caregiver Node user may touch a point on the map. In some cases, the geofencing software application responds by displaying a circle with a predetermined radius. The initial circle dimensions may be determined by coding in the Vitals™ Platform. In some cases, the Caregiver Node user may increase or decrease the circle's radius by, for example, manipulating the map, making a radius selection via a drop down menu, and/or in any other suitable manner. Once the radius size has been selected by the Caregiver Node, the area within the resulting circle can be selected by the Caregiver Node user and electronically marked as an Identified Zone. That designation may be stored in the Vitals™ Platform as part of the relevant Profile. Alternatively, in addition, the Caregiver Node user may be offered the option of using the map of the chosen location to touch relevant points or drag a finger to create lines, thereby “drawing” a boundary around a non-circular area on the map, such as a building's outer boundary, a campus, playground, or neighborhood. When the Caregiver Node indicates that the boundary selection is complete, the Caregiver Node can be used to select the chosen area as an Identified Zone rather than a circle having a selected radius.

In some cases, caregivers can use the Caregiver Node to create and store multiple simultaneous Identified Zones linked to a Beacon Node. In some instances, the Caregiver Node can be used to edit and/or delete Identified Zones as desired. The Identified Zones may be unique to each Beacon Node.

Upon creation of each Identified Zone, the Caregiver Node can be utilized to assign characteristics and notification attributes to each zone, sometimes expressed as “rules.” These rules can include, but are not be limited to, whether an Identified Zone is an authorized location or a prohibited off-limits location, whether notifications are always-on or only on during certain days and times, whether the same rules apply to all Identified Zone(s) and/or which rules apply to each Identified Zone, which receiving Nodes are authorized to receive which types of notification messages and under what circumstances and times, and/or specific methods of notification for each receiving Node (e.g. Email to Dad, text message to Grandma, both email and text message to Mom, current location and other information related to the vulnerable person to a 9-1-1 dispatch center).

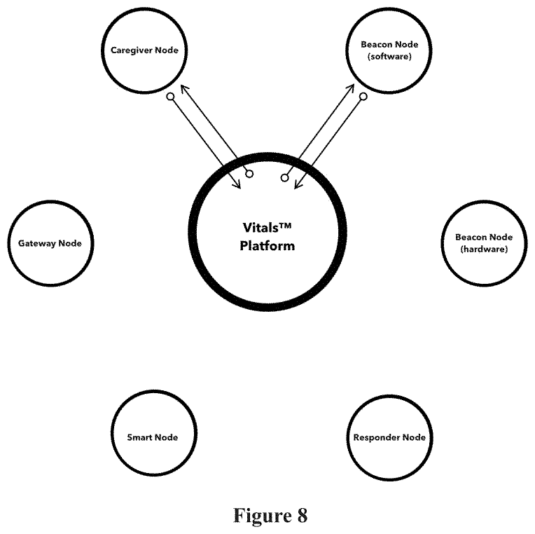

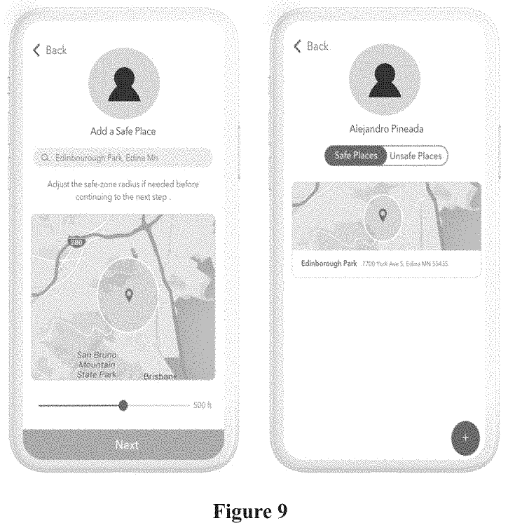

This geofencing function may allow caregivers to identify critical locations where their vulnerable charges should be, or should not be, and to provide real-time notifications. When the Caregiver Node adds any geofencing functions to the Profile, the specific geofencing information may be transmitted from the Vitals™ Platform to the relevant Beacon Node. This may occur only when a software Beacon Node is resident on a smart phone. FIG. 8 is a schematic block diagram of the system of FIG. 1 showing illustrative geo-fencing interactions among components of the networked system. FIG. 9 shows screen shots of an illustrative application program (app) running on a caregiver's mobile device showing an illustrative geo-fencing interaction feature.

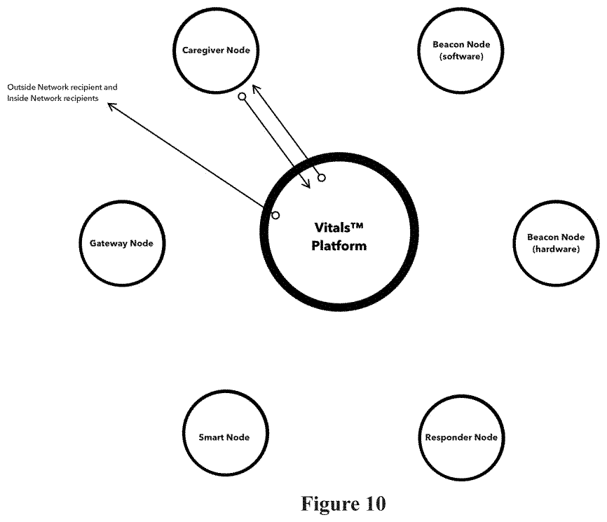

In some cases, the Caregiver Node can manually or automatically consolidate all of the current Profile, location and encounter information for a vulnerable person under the Caregiver Node's control. The consolidated information may be displayed on a “live” map of continually update GPS coordinates for the relevant paired smart phone Beacon Node(s). This may be referred to as a “god view.” The Caregiver Node also may enable access via a transmittable link to share the consolidated information with multiple other parties (sometimes subject to “rules” specified by the caregiver via the Caregiver Node). For example, the link could be sent to search parties, to first responders, or to 9-1-1 dispatch centers in support of an emergency call. The transmittable link may be shared directly with 9-1-1 dispatch centers that are not current users of the Vitals™ Network. FIG. 10 is a schematic block diagram of the system of FIG. 1 showing implementation of a “god view” functionality using components of the networked system.

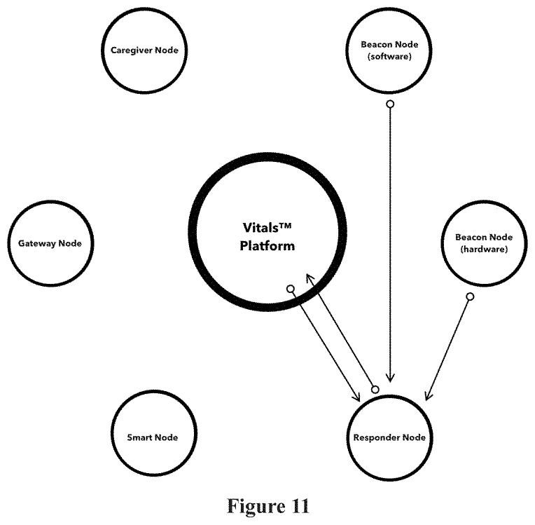

FIG. 11 is a schematic block diagram of the system of FIG. 1 showing illustrative responder interactions within the networked system via the Vitals™ Responder Node. As the name implies, the Vitals™ Responder Node is designed for use by professionals who interact with vulnerable persons and who would benefit from real-time delivery of certain critical information about the vulnerable persons whom they encounter. The users of the Responder Nodes would include but not be limited to law enforcement personnel, firefighters, emergency medical personnel, civilian security personnel, and professionals in such fields as education and health care. The Responder Node is intended for mobile environments.

The Responder Node may include, for example, a mobile phone running a Vitals™ Responder software application. The Vitals™ Responder software application may be created in multiple versions for all major operating systems of smart phones, and may be made available at suitable app stores on the Internet. The Responder Node may provide bidirectional communication with other nodes in the Vitals™ Network, all of which may be encrypted or otherwise encoded for security.

Inbound Vitals™ Beacon Node transmissions (e.g. coded BLE transmissions) are reliably detected by the Responder Node within approximately an 80-foot radius of the transmission source (e.g. Vitals™ Beacon Node). The Responder Node is intended to be on and active (so long as the host mobile device is on), and to be alert constantly for digital indications of other components of the Vitals™ Network. When the Responder Node detects the nearby presence of a Beacon Node (e.g. any beacon node registered with the Vitals™ Platform), the Responder Node retransmits the contents of the Beacon Node's unique identifier to the Vitals™ Platform using any available channel, including but not limited to WiFi, 4/5G, LTE cellular channels and/or any other suitable channel. The Responder Node may automatically adjust among channels to utilize the strongest signal and connection path.

The Vitals™ Platform may receive the coded retransmission from the Responder Node, identify the corresponding Profile, determine proper authorization(s), enable access to an appropriate database, select an appropriate unique dataset for the circumstances as specified in the Profile, and transmit that unique dataset to the Responder Node using WiFi, 4/5G, LTE cellular channels and/or any other suitable channel. The dataset may be a unique profile containing critical information relating to the particular vulnerable person that corresponds to the detected Beason Node (the corresponding “Profile”). Upon receipt of the Profile, the Responder Node may cause its host mobile device (e.g. smart phone) to generate predetermined alerts or notifications, which may include an audible tone, vibration, or visible effects on the smart phone's screen, or a combination of those. FIG. 3 depicts an example Profile as it might appear on the smart phone of a Responder Node. The Responder Node user, sometimes through a Vitals™ Responder software application, may control which types of alerts or notifications are presented but it may not entirely suppress the notification itself. The downloaded Profile may be displayed on the screen of a smart phone of the Responder Node to provide critical information to the user of the Responder Node.

The Responder Node may be designed to handle an unlimited number of Profile deliveries, which may appear on the smart phone's screen in list (or other) format for selection by the user. Although the Responder Node may be always on and always operational, sometimes in the background, a user may mute alerts during off-duty times or at other times when silence may be required.

Upon sensing the presence of a registered Beacon Node signal, the Responder Node may transmit to the Vitals™ Platform a unique identifying signal, as well as GPS coordinates. This may occur in both foreground and background mode. Utilizing the always-on nature of the Responder Node, whenever it detects the presence of another component of the Vitals™ Network, the Responder Node may utilize WiFi, cellular and/or any other available channels to convey the encounter information to the Vitals™ Platform, including the GPS coordinates of the transmitting Responder Node. So long as the mobile device is powered on and regardless of whether the device is muted, information still may be gathered by the Responder Node and shared with the Vitals™ Platform.

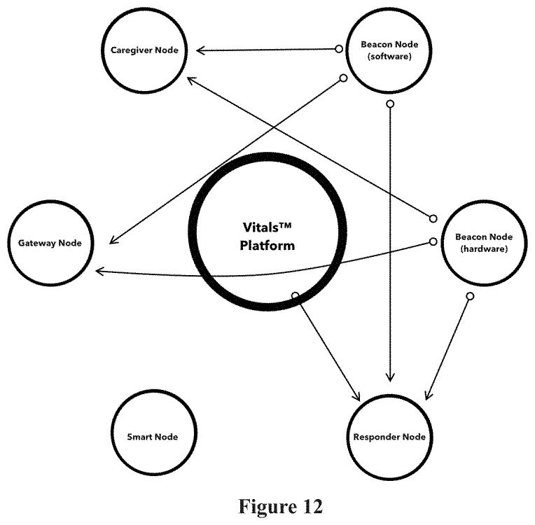

FIG. 12 is a schematic block diagram of the system of FIG. 1 showing illustrative beacon node interactions of networked components of the system. The Beacon Node is intended for use by a vulnerable person and its function may be primarily to gather and transmit information. At frequent intervals, the Beacon Node may transmit an omnidirectional timed signal containing its unique identifying code. If the Beacon Node is a physical device including a battery, sometimes the transmitted omnidirectional timed signal will include information regarding estimated remaining battery life. In some cases, the Beacon Node may correspond to a smart phone and/or smart watch running a Vitals™ Beacon software application.

In some cases, the Beacon Node may be implemented as an inexpensive physical device that can be provided to the vulnerable person in a variety of useful forms such as a lanyard, a credit card size device, a small plastic square or disc, a device that is configured to be secured to a vulnerable persons shoe and/or shoe laces, etc. (These are just examples.) The Beacon Node may include a suitable chipset and a battery. The chipset mass may be small, and greatest bulk of the mass may be in the battery. The beacon may simply transmit a unique identifier via BLE or the like, and in some cases, information regarding estimated remaining battery life. Because such a device would be expected to have relatively low energy requirements, battery life would be expected to range from 3 to 18 months. During an encounter, the information regarding estimated remaining battery life may be received by the Responder Nodes and/or Caregiver Nodes and transmitted to the Vitals™ Platform. In some cases, the Vitals™ Platform may automatically cause a replacement beacon device to be shipped to the Profile address in advance of battery expiration, and to warn caregivers of pending battery failure. The Beacon Node may be designed to be always on. In some case, the Beacon Nodes cannot be powered off.

During normal use, the Beacon Node may prioritize BLE for its omnidirectional frequent transmissions at regular intervals. However, the Beacon Nodes may be configured to automatically choose from among available communication channels, including but not limited to WiFi, 4/5G or LTE cellular, as desired.

When the Beacon Node is a software application resident in a smart phone, the Vitals™ Platform geofencing functionality can be activated. When the Caregiver Node has added geofencing instructions to a Profile, the relevant geofencing information may be transmitted from the Vitals™ Platform to the smart phone Beacon Node. When the transmission has been received by the smart phone Beacon Node, the software application of the smart phone Beacon Node may cause the smart phone Beacon Node to begin to detect the phone's location via the smart phone Beacon Node's location services. If and when the smart phone Beacon Node intersects geographically with an Identified Zone, the smart phone Beacon Node may immediately transmit the time, location and/or other information to the Vitals™ Platform. For these transmissions of time, location and/or other information, the smart phone Beacon Node may automatically adjust among available channels, including but not limited to WiFi, 4/5G, LTE cellular and/or any other available channel. Upon receipt, identification and authentication of the location transmission from the smart phone Beacon Node, the Vitals™ Platform may follow pre-determined notification instructions/rules contained in the relevant Profile. In some cases, recipients of notifications can be customized by the relevant Caregiver Node and can include, for example, caregivers, other family members, guardians, responders, administrators, etc.

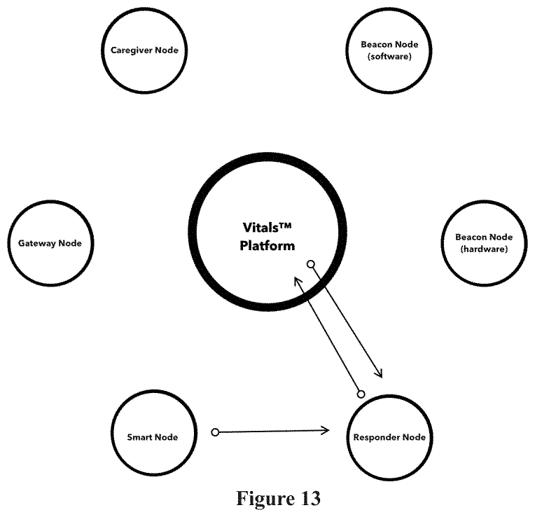

FIG. 13 is a schematic block diagram of the system of FIG. 1 showing illustrative smart node interactions of components within the networked system. A Smart Node may be a physical device designed to be temporarily or permanently installed in locations chosen to enhance functionality of the Vitals™ Network by monitoring for registered Beacon Nodes in their typical locations (homes, schools, medical facilities, etc.). The Smart Node may be designed to be plugged into standard electrical outlets and may operate on normal household electrical power to enhance likelihood of consistent availability. The Smart Node may utilize bidirectional transmission capabilities and in some cases may be designed to select a digital transmission channel that has the strongest available signal. When supplied with electrical power, the Smart Node may function much as the Responder Node discussed herein, detecting the proximity of registered Beacon Nodes. In some cases, the proximity information is detected, received, assembled, and transmitted in real or near real time to the Vitals™ Platform. The Smart Node may act as an information concentrator. A Caregiver Node may be configured to setup and control a Smart Node. A configuration may be created for the location of the Smart Node, identifying relevant Beacon Nodes that are authorized to be detected within range of the Smart Node's location (e.g. approximately 80 feet). The transmission of information from the Smart Node may include information on authorized Beacon Nodes as well as information on Beacon Nodes that actually are within proximity detection range at a given time, allowing identification of missing Beacon Nodes.

Smart Nodes may be used in, for example, group housing and institutional settings. The Smart Node will be of use in emergency situations when vulnerable persons must be accounted for. Smart Nodes may also enable more granular information gathering, and by increasing network density by providing more Smart Nodes, richer sets of information over time may be generated that may be useful for security and caregiving purposes. This may allow more accurate location detection for Beacon Nodes.

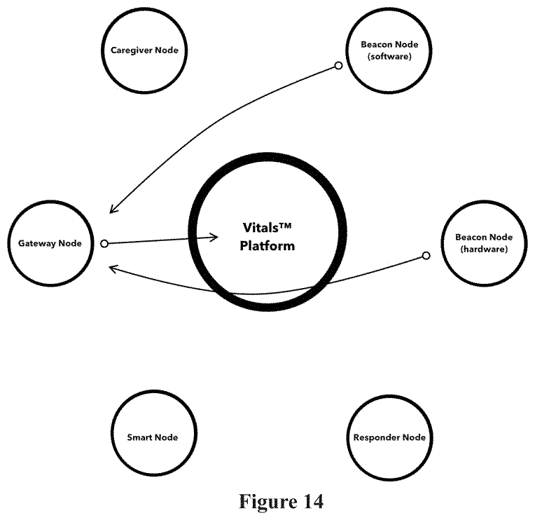

FIG. 14 is a schematic block diagram of the system of FIG. 1 showing illustrative gateway node interactions of components within the networked system. In some cases, a Gateway Node may operate on household electrical power to enhance likelihood of consistent functionality, and may utilize bidirectional transmission capabilities. When supplied with electrical power, the Gateway Node may function much as the Responder Node, detecting the proximity of Beacon Nodes and in real or near real time transmitting the Beacon Node information to the Vitals™ Platform for recording and processing.

Like the Smart Nodes, the Gateway Node may automatically adjust among available communication channels including but not limited to WiFi, 4/5G, LTE cellular and/or any other available channel to utilize the strongest signal and connection path. Gateway Node may be used in such locations as public transit locations, mall entrances, theaters, stadiums and event centers, as well as in schools and other locations where appropriate. The Gateway Node may focus on detecting transitory physical encounters and in providing enhanced monitoring capabilities in geographical locations that are more likely to cause distress or difficulty for vulnerable persons. The Gateway Nodes may enable another form of location detection with more granular information gathering, and by increasing network density they may generate richer sets of information that are useful for security and caregiving purposes. Greater location granularity provides more accurate location detection for Beacon Nodes.

In some cases, an illustrative method of assembling, managing and transmitting pre-defined information from one user to one or more other users in real time using multiple and selectable transmission media in secure storage and transmission environments may include: receiving information into the platform from multiple sources, categorizing it into pre-determined types, and defining authorized recipients of the information; enabling authorized users to instruct the platform to change the information and to change authorizations for receipt of the information; activating secure bi-directional devices that receiving users (e.g. first responders) and that passive users (e.g. vulnerable persons) utilize to receive and to transmit signals intended for ultimate receipt by other users; causing all communications from all user devices to pass into, through, and out of the platform; enabling contributing users (e.g. caregivers) to control who receives information related to the contributing user's (e.g. caregivers) relevant vulnerable person, including but not limited to maintenance by the platform for the contributing user (e.g. caregivers) of authorized recipients and disallowed recipients; and capturing and storing all information derived from activities of the platform (e.g. logging events and/or activities of the users).

Ins some cases, the illustrative method may include receiving user-defined information into the platform from contributing users (e.g. caregivers), inserting the contributed information into a secure database in which the contributing user (e.g. caregiver) maintains access to and sole control of the contributed information; managing and organizing the information into pre-determined categories, each category to define the type of recipient for that category's information, and allowing the contributing user (e.g. caregiver) to control and alter the criteria for categorization; equipping receiving users (e.g. first responders) with secure bi-directional devices that, communicating through the platform, detect other users (e.g. vulnerable persons) within close proximity of the receiving user's (e.g. first responders) device and, upon detection, to transmit that detection to the platform; causing the platform to respond to the notice of detection, to ascertain authorization levels and to apply the pre-determined criteria to select the information in accordance with pre-determined categorization, and to deliver that selected information to the receiving user (e.g. first responder); and equipping passive users (e.g. vulnerable persons) with secure bi-directional devices that emit recurring intermittent coded signals that identify the passive user (e.g. vulnerable person) possessing the device.

In some cases, the illustrative method may include providing the contributing user's (e.g. caregiver's) device with functions enabling transmission of signals through the platform and ultimately delivered to authorized passive user (e.g. vulnerable person) devices such that, when received, the signals cause the passive user's (e.g. vulnerable person) device to respond with a pre-determined set of information transmitted to the platform and then transmitted to the contributing user's (e.g. caregiver's) device; and providing the passive user's (e.g. vulnerable person's) device with functions to respond to signals received from the authorized contributing user (e.g. caregiver), such responses to include but not be limited to newly created information generated in real time and providing information about location, status and/or other facts.

In some cases, the illustrative method may include providing the contributing user's (e.g. caregiver's) device with the ability to pair electronically with one or more passive user (e.g. vulnerable person) devices, said pairing to be under the sole control of the contributing user's (e.g. caregiver's) device and to occur by communicating through the platform; providing the contributing user's (e.g. caregiver's) device with the ability to communicate through the platform to activate or to deactivate recurring intermittent signals sent from the contributing user's (e.g. caregiver's) device to the platform and then onward to one or more selected passive user (e.g. vulnerable person) devices, and for the passive user (e.g. vulnerable person) devices to activate recurring intermittent signals sent from the passive user (e.g. vulnerable person) devices to the platform and ultimately transmitted onward to the contributing user's (e.g. caregiver's) device; and during activation as authorized by the contributing user's (e.g. caregiver's) device, enabling the platform to monitor all paired devices, to ascertain proximity to each other, and to notify the user's device in real time when proximity has been interrupted or has ceased.

In some cases, the illustrative method may further include providing the contributing user (e.g. caregiver) with abilities to specify one or more geographical points or boundaries and to have the platform add those geographical points or boundaries to the passive user's (e.g. vulnerable person) profile; enabling the contributing user (e.g. caregiver) to communicate with the platform to activate the geofencing function associated with the passive user's (e.g. vulnerable person) profile; causing the passive user's (e.g. vulnerable person) device to detect any physical intersection with or crossing of any boundary stored in the profile associated with the passive user (e.g. vulnerable person); causing any such detection to generate a transmission to the platform, such transmission containing information including, but not limited to, time and date and geographical location; and causing the platform to convey such transmission in real time onward to the contributing user's (e.g. caregiver's) device.

The illustrative method may further include providing that all components of the platform coordinate the components' actions to provide real-time information in accordance with rules created and assigned to each passive user's (e.g. vulnerable person) profile.

What is claimed is:

1. A method for delivering timely personalized information of vulnerable persons to authorities and/or caregivers, the method comprising:
receiving an identifier for a vulnerable person from a first remote device that has come within range of and detected a beacon of the vulnerable person;
using the identifier to identify the vulnerable person;
identifying a profile for the identified vulnerable person, wherein at least part of the profile has been provided by a second remote device;
providing at least part of the identified profile to the first remote device that has come within range of and detected the beacon of the vulnerable person; and
sending a notification to the second remote device when the first remote device has come within range of and detected the beacon of the vulnerable person.

2. The method of claim 1 wherein the first remote device is a remote device of a first responder, and the second remote device is a remote device of a caregiver of the vulnerable person.

3. The method of claim 1, wherein the beacon repeatedly transmits a beacon identifier signal using Bluetooth Low Energy (BLE), and the first remote device receives the beacon identifier signal when the first remote device is within range of the beacon.

4. The method of claim 3, wherein the first remote device transmits the identifier, wherein the identifier is based at least in part on the beacon identifier signal.

5. The method of claim 1, wherein the receiving, using, identifying, providing and sending steps are performed by a server.

6. The method of claim 5, wherein the server uses the identifier to identify the vulnerable person by indexing the identifier into a vulnerable person database.

7. The method of claim 6, wherein the server identifying the profile for the identified vulnerable person by indexing into the vulnerable person database.

8. The method of claim 7, wherein the second remote device communicated with the server to provide at least part of the profile, wherein the server receives and enters the profile into the vulnerable person database.

9. A method for delivering timely personalized information of vulnerable persons to authorities and/or caregivers, the method comprising:
receiving an identifier for a vulnerable person from a first remote device that has come within range of and detected a beacon of the vulnerable person;
using the identifier to identify the vulnerable person;
identifying a profile for the identified vulnerable person, wherein at least part of the profile has been provided by a second remote device;
providing at least part of the identified profile to the first remote device that has come within range of and detected the beacon of the vulnerable person;
detecting that the beacon has crossed a predefined geofence; and
sending a notification to the second remote device when the beacon has crossed the predefined geofence.

10. The method of claim 9, wherein the notification to the second remote device includes location information of the beacon.

11. The method of claim 9, further comprising receiving location information of the beacon, and transmitting the location information to a 9-1-1 dispatch center.

12. The method of claim 11, further comprising receiving an alert from the second remote device after sending the notification to the second remote device when the beacon has crossed the predefined geofence, the alert triggering the transmitting of the location information to a 9-1-1 dispatch center.

13. A method for delivering timely personalized information of vulnerable persons to authorities and/or caregivers, the method comprising:
receiving an identifier for a vulnerable person from each of a plurality of responder remote devices over time that have encountered the vulnerable person by coming within range of and detecting a registered beacon of the vulnerable person;
using the identifier to identify the vulnerable person on each encounter;
identifying a profile for the identified vulnerable person on each encounter, wherein at least part of the profile has been provided by a caregiver remote device;
providing at least part of the identified profile to the corresponding responder remote device(s) associated with each encounter; and
sending a history of the encounters to the caregiver remote device.

14. The method of claim 13, wherein the history of the encounters is sent upon receiving a request from the caregiver remote device.

15. The method of claim 14, wherein the history of the encounters can be filtered to only send part of the encounters to the caregiver remote device.

16. A non-transient computer readable medium storing instructions that when executed by one or more processors causes the one or more processors to:
allow each of a plurality of caregivers to register a corresponding vulnerable person, wherein registering each vulnerable person includes:
receiving a unique identifier of a beacon carried or worn by the corresponding vulnerable person;
receiving a profile for the corresponding vulnerable person that identifies a vulnerability of the corresponding vulnerable person;
receiving contact information of a caregiver of the corresponding vulnerable person;
receive an acquired identifier acquired by a remote device of a first responder that has come within range of and detected a beacon of a vulnerable person in the field during an encounter;
match the acquired identifier received from the remote device of the first responder with a unique identifier of a registered vulnerable person, and sending at least part of the profile of the matched registered vulnerable person to the remote device of the first responder; and
send a notification to the caregiver associated with the vulnerable person during the encounter.

17. The non-transient computer readable medium of claim 16, wherein the instructions further cause the one or more processors to facilitate communication between the remote device of the first responder and the caregiver associated with the vulnerable person.

18. The non-transient computer readable medium of claim 17, wherein communication between the remote device of the first responder and the caregiver associated with the vulnerable person is established through a system that includes the one or more processors.

19. The non-transient computer readable medium of claim 16, wherein the instructions cause the one or more processors to receive a notification from the caregiver associated with the vulnerable person of a location of the vulnerable person to initiate the encounter with the vulnerable person.

20. The non-transient computer readable medium of claim 19, wherein the instructions cause the one or more processors to send the location in the notification from the caregiver associated with the vulnerable person to remote device of the first responder to initiate the encounter.
